SEQUENCE LISTING (9 sequences)

SEQ ID NO: 1 - DNA, 255 bp, Rattus norvegicus
gggaggagcgaccgacacgaactagaaattatgagcacaagtacactggaatcctccagcttcagagctgcttcctccacccacagacctgcttcctcctccaccgtgcccgagcaggcctaacctccagaccgtcagagaggacagctatggtctaggacagttctggtgttaccctggagtccacgggaggggaactagtccagactgcctgagatgagtaaagtatctggcgtcaccaaaaaaaaaaaaaaa

SEQ ID NO: 2 - DNA, 1387 bp, Rattus norvegicus
ctcactttatctggggtgcggatcgggtcaagaggttgaaggtgctgtgcgtgatccaggatccaagttggcccccggagcaggagcatggcgtcgtcgggatcggtgcagcagctgcggctggtgctgctgatgttgctgctggccggtgcggcacgggccagcctctacttccgcccgggccagacctgctaccgaccccttcacagggaccacctggctctgctggggcgcaggacctatcctcggccacatgagtacctgtccccagcggatctccccaagaactgggactggaggaatgtgaacggtgtcaactatgccagcgtcacgaggaatcagcatatcccacagtactgtggttcttgctgggcccacggcagcaccagtgccttggcagatcgcatcaacatcaagagaaaaggtgcatggccctccaccctgttgtcggtgcagaatgtcatcgactgcggcaatgctggttcttgtgaggggggcaatgaccttccggtgtgggagtatgcccacaagcatggcatccccgatgagacctgcaacaactaccaggccaaggaccaagaatgtgacaagtttaaccagtgtgggacctgcactgaattcaaagagtgccacaccatccagaattataccctctggagagtgggtgactatggctccctgtccgggagggagaagatgatggcagagatctacgccaatggtcccatcagctgcgggataatggcaaccgagaggatgtctaactacactggaggcatctatactgagtaccagaaccaggccattatcaaccacatcatctctgtagccggctggggtgtcagcaatgatggcatcgagtattggattgtccgcaattcatggggcgagccctggggtgagcgaggctggatgagaattgtgaccagcacctataagggagggacaggttccagctacaacctcgccattgaggaggcctgcacatttggggaccccattgtctaggtagatgtccctggaagcaacactgtgaaccatgatgaggaggggtgattatcgacactggatatgtccattcagctagaaacagtggccctcatgtggacacgaggaccagagtgtgggctgcaccccgagaggtgataggaaaggatgagccacaactacactggaaccctccaccttcagacctgcttccctccacccacagacctgcttcctcctccaccgtgccctgcaggcctacctccagccgccagagaggacagctatggtctaggacagttctggtgttaccctggagtccacgggaggggaactagtccagactgcctgagatgagtaaagtatctggcgtcaccaaaaaaaaaaaaaaaaaaaaaaa

SEQ ID NO: 3 - protein, 306 aa, Rattus norvegicus
MASSGSVQQLRLVLLMLLLAGAARASLYFRPGQTCYRPLHRDHLALLGRRTYPRPHEYLSPADLPKNWDWRNVNGVNYASVTRNQHIPQYCGSCWAHGSTSALADRINIKRKGAWPSTLLSVQNVIDCGNAGSCEGGNDLPVWEYAHKHGIPDETCNNYQAKDQECDKFNQCGTCTEFKECHTIQNYTLWRVGDYGSLSGREKMMAEIYANGPISCGIMATERMSNYTGGIYTEYQNQAIINHIISVAGWGVSNDGIEYWIVRNSWGEPWGERGWMRIVTSTYKGGTGSSYNLAIEEACTFGDPIV

SEQ ID NO: 4 - DNA, 1500 bp, Homo sapiens
ggggtcggccgggtgctaggccggggccgaggccgaggccggggcgggatccagagcgggagccggcgcgggatctgggactcggagcgggatccggagcgggacccaggagccggcgcggggccatggcgaggcgcgggccagggtggcggccgcttctgctgctcgtgctgctggcgggcgcggcgcagggcggcctctacttccgccggggacagacctgctaccggcctctgcggggggacgggctggctccgctggggcgcagcacatacccccggcctcatgagtacctgtccccagcggatctgcccaagagctgggactggcgcaatgtggatggtgtcaactatgccagcatcacccggaaccagcacatcccccaatactgcggctcctgctgggcccacgccagcaccagcgctatggcggatcggatcaacatcaagaggaagggagcgtggccctccaccctcctgtccgtgcagaacgtcatcgactgcggtaacgctggctcctgtgaagggggtaatgacctgtccgtgtgggactacgcccaccagcacggcatccctgacgagacctgcaacaactaccaggccaaggaccaggagtgtgacaagtttaaccaatgtgggacatgcaatgaattcaaagagtgccacgccatccggaactacaccctctggagagtgggagactacggctccctctctgggagggagaagatgatggcagaaatctacgcaaatggtcccatcagctgtggaataatggcaacagaaagactggctaactacaccggaggcatctatgccgaataccaggacaccacatatataaaccatgtcgtttccgtggctgggtggggcatcagtgatgggactgagtactggattgtccggaattcatggggtgaaccatggggcgagagaggctggctgaggatcgtgaccagcacctataaggatgggaagggcgccagatacaaccttgccatcgaggagcactgtacatttggggaccccatcgtttaaggccatgtcactagaagcgcagtttaagaaaaggcatggtgacccatgaccagaggggatcctatggttatgtgtgccaggctggctggcaggaactggggtggctatcaatattggatggcgaggacagcgtggtactggctgcgagtgttcctgagagttgaaagtgggatgacttatgacacttgcacagcatggctctgcctcacaatgatgcagtcagccacctggtgaagaagtgacctgcaacacaggaaacgatgggacctcagtcttcttcagcagaggacttgatattttgtatttggcaactgtgggcaataatatggcatttaagaggtgaaagagttcagacttatcaccattcttatgtcactttagaatcaagggtgggggagggagggagggagttggcagtttcaaatcgcccaactgataaataaagtatctggctctgcacgag

SEQ ID NO: 5 - protein, 240 aa, Homo sapiens
MARRGPGWRPLLLLVLLAGAAQGGLYFRRGQTCYRPLRGDGLAPLGRTTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYIL

SEQ ID NO: 6 - protein, 11 aa, Artificial Sequence
  oligopeptide
LFYDQSPTATI

SEQ ID NO: 7 - protein, 12 aa, Artificial Sequence
  oligopeptide
YKRDMVGGVVIA

SEQ ID NO: 8 - protein, 11 aa, Artificial Sequence
  oligopeptide
EGYYGNYGVYA

SEQ ID NO: 9 - protein, 12 aa, Artificial Sequence
  oligopeptide
FFDEPNPGVTIY

(19) United States
(12) Patent Application Publication
(10) Pub. No.: US 2003/0232740 A1
(43) Pub. Date: Dec. 18, 2003

(54) Cathepsin Y for the development of a medicament for the treatment of stroke

(76) Inventors: Hermann Lubbert, Leverkusen (DE); Stefan Zwilling, Leverkusen (DE); Peter Engels, Bergisch Gladbach (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/392,809

(22) Filed: Mar. 19, 2003

Related U.S. Application Data
(60) Provisional application No. 60/366,337, filed on Mar. 20, 2002.

(51) Int. Cl. A61K 31/00; C12Q 1/68; G01N 33/53; G01N 33/567

(52) U.S. Cl. 514/1; 435/6; 435/7.2

(57) ABSTRACT

The present invention relates to the use of a polynucleotide sequence en-coding Cathepsin Y or a amino acid sequence of Cathepsin Y protein for the characterization or identification of therapeutic agents for stroke, the use of such sequences for the development of a medicament, and agents useful as medicaments for the treatment of stroke.

### RELATED APPLICATION

This application claims priority under 35 U.S.C. § 119(e) to U.S. Provisional Application 60/366,337, filed Mar. 20, 2002.

### FIELD OF THE INVENTION

The present invention relates to the use of a polynucleotide sequence encoding Cathepsin Y or an amino acid sequence of Cathepsin Y protein for the characterization or identification of therapeutic agents for stroke, the use of such sequences for the development of a medicament, and agents useful as medicaments for the treatment of stroke.

### BACKGROUND OF THE INVENTION

Stroke is a rather common and potentially harmful event that often leaves patients severely disabled for the rest of their lives. It is the result of either an interruption of blood and therefore oxygen supply to the brain, or bleeding in the brain that occurs when a blood vessel bursts. The reduced oxygen supply leads to shortage of energy in the affected brain regions and results in the death of neurons around the lesion. The degeneration spreads from the site that is affected by the reduced blood supply into regions which have at all times obtained sufficient oxygen.

Stroke results from a loss of blood flow to the brain caused by thrombosis or haemorrhage. With an incidence of 250-400 in 100,000 and a mortality rate of around 30%, stroke is a major public health problem. About one-half of the stroke survivors suffer from significant persisting neurological impairment and/or physical disability. Thus, the economic costs of stroke amount to many billions of dollars worldwide.

A stroke occurs when the blood supply to part of the brain is suddenly interrupted or when a blood vessel in the brain bursts. As a consequence, brain cells die when they no longer receive oxygen and nutrients from the blood or when they are damaged by sudden bleeding into the brain. Some brain cells die immediately after interruption of the blood flow into the brain, while others remain at risk for death and stay in a compromised state for hours. These damaged cells make up the so-called “ischemic penumbra”, and with timely treatment these cells could be saved. Stroke ultimately leads to infarction, the death of huge numbers of brain cells, which are eventually replaced by a fluid-filled cavity (or infarct) in the injured brain.

There are two major forms of stroke: ischemic—blockage of a blood vessel supplying the brain, and hemorrhagic—bleeding into or around the brain. An ischemic stroke can be caused by a blood clot (embolus or thrombus), which is blocking a vessel. It can also be caused by the narrowing of an artery due to the build-up of plaque (a mixture of fatty substances, including cholesterol and other lipids). The underlying pathological process called stenosis is often observed in arteriosclerosis, the most common blood vessel disease. About 80% of all strokes are ischemic strokes. A hemorrhagic stroke is caused by the bursting of an artery in the brain. Subsequently, blood spews out into the surrounding tissue and upsets not only the blood supply but also the delicate chemical balance neurons require to function. Hemorrhagic strokes account for approximately 20% of all strokes.

A transient ischemic attack (TIA), sometimes called a mini-stroke, starts just like a stroke but then resolves leaving no noticeable symptoms or deficits. The occurrence of a TIA is a warning that the person is at risk for a more serious and debilitating stroke. About one-third of patients who have a TIA will have an acute stroke sometime in the future. The addition of other risk factors compounds a person's risk for a recurrent stroke. The average duration of a TIA is a few minutes. For almost all TIAs, the symptoms go away within an hour. There is no possibility to distinguish whether symptoms will be just a TIA or persist and lead to death or disability.

Recurrent stroke is frequent; about 25 percent of people who recover from their first stroke will have another stroke within 5 years. Recurrent stroke is a major contributor to stroke disability and death, with the risk of severe disability or death from stroke increasing with each stroke recurrence. The risk of a recurrent stroke is greatest right after a stroke, with the risk decreasing with time. About 3 percent of stroke patients will have another stroke within 30 days of their first stroke and one-third of recurrent strokes take place within 2 years of the first stroke.

The most important risk factors for stroke are hypertension, arteriosclerosis, heart disease, diabetes, and cigarette smoking. Others include heavy alcohol consumption, high blood cholesterol levels, illicit drug use, and genetic or congenital conditions, particularly vascular abnormalities. People with multiple risk factors compound the destructive effects of these risk factors and create an overall risk greater than the simple cumulative effect of the individual risk factors.

Although stroke is a disease of the brain, it can affect the entire body. Depending on the affected brain region and the severity of the attack, post-stroke patients suffer from a variety of different symptoms. Some of the disabilities that can result from stroke include paralysis, cognitive deficits, speech problems, emotional difficulties, daily living problems, and pain. Stroke disability is devastating to the stroke patient and family, but therapies are available to help rehabilitate post-stroke patients. The mortality rate observed in ischemic stroke is around 30%. The time window for a medical treatment is narrow and limited to anticoagulants and thrombolytic agents, which must be given immediately (at latest 3 hours) after having a stroke. Unfortunately, there is no effective neuroprotective medication available, which is able to stop the delayed degeneration of neurons following the initial stroke attack.

Stroke symptoms appear suddenly. The following acute symptoms can be observed. Sudden numbness of the face, arm or leg, difficulties in talking or understanding speech, trouble seeing in one or both eyes, sudden trouble in walking, loss of balance and coordination. Severe headache with no known cause does also occur. Even more importantly, there is a variety of severe disabilities occurring and persisting in post-stroke patients. Paralysis is a frequent disability resulting from stroke. Cognitive deficits (problems with thinking, awareness, attention and learning) are also commonly observed. Post-stroke patients exhibit language deficits and also emotional deficits (like post-stroke depression). Furthermore, an uncommon type of pain, called central pain syndrome (CPS), can occur after having a stroke.

Currently the only effective treatment for thrombotic stroke is the use of anticoagulants (e.g. heparin), and thromobolytics (recombinant tissue plasminogen activator). Neuroprotective agents, which are effective in animal models, have generally proved ineffective in the clinic, and none are yet registered for use in stroke.

A number of experimental models have been developed for global ischemia and focal ischemia. The availability of these different models provides an opportunity to investigate mechanisms of stroke. Finding common features in different models or over several time points within one model should pro-vide better insight into the mechanisms critical for stroke, and comparison of the models should help to understand development, progression and consequences of stroke.

Most common global ischemia models are

a) the two vessel model

Models of transient global ischemia resulting in patterns of selective neuronal vulnerability are models that attempt to mimic the pathophysiology of cardiac arrest or hemodynamic conditions that result from severe systemic hypotension. Reversible high-grade forebrain ischemia is generated by bilateral common carotid artery (CCA) occlusion. Together with systemic hypo-tension conditions it reduces the blood flow to severe ischemic levels (Smith et al. 1984 Acta Neuropathol 64:319-332). This model of transient global ischemia has the advantage of a one stage surgical preparation, the production of a high-grade forebrain ischemia and the possibility to conduct chronic survival studies in order to assess the potential of neuroprotective drugs.

b) the four vessel model

This model results in a high-grade forebrain ischemia but is produced in two stages, one to manipulate each of the CCA, and the second stage 24h later to produce the forebrain ischemia. The advantage is that the second step can be produced in awake freely moving animals (Pulsinelli et al. 1982 Ann Neurol 11:491-498). Similar pathohistological results could be obtained for both vessel models.

c) the cardiac arrest model

Forebrain ischemia models are of value to study cerebral ischemia but these models do not exactly mimic the hemodynamic consequences of a cardiac arrest, which results in a complete ischemia of the brain, spinal cord and extracerebral organs (Katz et al. 1995 J Cereb Blood Flow Metab 15:1032-1039). The initial cardiac arrest models from Safar et al. (1982 Protection of tissue against hypoxia Elsevier Biomedical Press; 147-170) or Korpaczew et al. (1982 Partol Fizjol Eksp Ter 3:78-80), were developed further by Katz et al. (1989 Resuscitation 17:39-53) and Pluta et al. (1991 Acta Neuropathologica 83:1-11) to models with controllable insult. Katz and colleagues (1995) have reported a reproducible outcome model of cardiac arrest with apneic asphyxia of 8 min, leading to the cessation of circulation at 3-4 min of apnea and resulting in cardiac arrest of 4-5 min. At 72 hr after injury, widespread patterns of ischemic neurons were found in many brain regions, including cerebral cortex, caudate putamen CA1 and CA3 regions of hippocampus, thalamus, cerebellum and brain stem.

Pluta et al. described a primary mechanical cardiac arrest model whereby global ischemia was induced by cardiac arrest for 3 to 10 min with survival periods of the animals from 3 min to 7 days.

Although these models have several limitations, they provide a method for studying the mechanisms of neuronal injury resulting from the clinically realistic cerebral insult and screening potential cerebral resuscitation therapies.

Focal Ischemia models

Models of permanent or transient focal ischemia typically giving rise to localized brain infarction have routinely been used to investigate the pathophysiology of stroke. For example, models of middle cerebral artery (MCA) occlusion in a variety of species have gained increased acceptance due to their relevance to the human clinical setting.

a) the permanent MCA Occlusion models

Tamura and colleagues (1981 J Cereb Blood Flow Metab 1:53-60) developed a subtemporal approach as standard model of proximal MCA occlusion. In models of permanent MCA occlusion, electrocauterization of the MCA proximal to the origin of the lateral lenticulostriate arteries is utilized routinely. In these models, severe reductions in blood flow are seen within the ischemic core, with milder reductions in blood flow within the border or penumbral regions. The addition of moderate arterial hypotension has the effect of enlarging infarct volume.

b) the transient MCA Occlusion models

In human ischemic stroke, recirculation frequently occurs after focal ischemia. Thus, models of transient MCA occlusions have also been developed, mainly in rats or mice, whereby surgical clip or sutures are introduced to induce a transient ischemic insult.

Some further animal models for stroke are considered in several reviews like the articles of W. D. Dietrich (1998 Int Review of Neurobiology 42:55-101) , Wiebers et al. (1990 Stroke 21:1-3) or Zivin and Grotta (1990 Stroke 21:981-983).

### SUMMARY OF THE INVENTION

Object of the present invention is to provide a target for examination and development of a medicament for the treatment of stroke.

This object is met by a method for the development of a medicament for the treatment of stroke comprising the use of a polynucleotide sequence encoding Cathepsin Y or homologues or fragments thereof or the according poly-peptide.

According to the invention it has been found that Cathepsin Y is differentially expressed under stroke, particularly under ischemic stroke. In the present invention it is shown that in the rat models Cathepsin Y expression is upregulated under stroke, particularly under global ischemia in the cardiac arrest model (Pluta et al. 1991 Acta Neuropathol 83: 1-11).

The model is explained in more detail in the literature and in the examples below.

### BRIEF DESCRIPTION OF THE DRAWINGS

FIG. 1 shows Cathepsin Y transcript differentially expressed over several time points in the cardiac arrest model. Cathepsin Y is examined in its expression level over a time period of 0.5 hrs after cardiac arrest to 2 years after surgery and compared to sham operated controls. Cathepsin Y is described as differentially expressed when the sequence is up or down regulated at one or more time points with a certain statistical relevant significance value (Δh>±0.2).

X-axis describes the time points analyzed by DEPD, 1=0.5 h post operation, 2=1 hr post operation, 3=3 hrs post operation, 4=6 hrs post operation, 5=day 3 post operation, 6=day 7 post operation, 7=2 years post operation.

The Y-axis shows Δh, which represents the normalized difference of expression (peak height) of Cathepsin Y between a control group and a treated group. x-fold difference in gene expression is calculated by

$$\frac{1 + Δ h}{1 - Δ h}$$

0=no change to control, +=up regulation, −=down regulation; (0.2=1.5 fold; 0.3=1.86 fold; 0.4=2,33 fold; 0.5=3 fold expression level).

### DETAILED DESCRIPTION OF THE PREFERRED EMBODIMENT

The term “polynucleotide sequence” or “nucleic acid sequence” designates in the present application any DNA or RNA sequence, independent of the length. Thus this term can describe short sequences like PCR primers or probes for hybridization, as well as whole genes or cDNA of these genes.

The term “polypeptide” or “amino acid sequence” designates a chain of amino acids, independent of their length, however, in any case more than one amino acid.

As “homologues” of polynucleotide sequences such polynucleotide sequences are designated which encode the same type of protein as one of the polynucleotide sequences described herein. Accordingly as “homologues” of a poly-peptide the polypeptides are designated, which have an amino acid sequence, wherein at least 70%, preferably 80%, more preferably 90% of the amino acids are identical to one of the proteins of the present invention and wherein the replaced amino acids preferably are replaced by homologous amino acids. As “homologous” amino acids are designated those, which have similar features concerning hydrophobicity, charge, steric features etc. Most preferred are amino acid sequences, containing the species- or family-dependent differences of the amino acid sequence. Particularly as “homologues” sequences are designated which correspond to one of the cited sequences in another species or individual. For example if in the present invention a rat model is used and the cited polynucleotide sequence encodes the rat protein, the according polynucleotide sequence and protein of a mouse in a mouse model is designated as “homologue”. Further splice variants and members of gene families are designated as homologues.

“Fragments” of a polynucleotide sequence are all polynucleotide sequences, which have at least 10 identical base pairs compared to one of the polynucleotide sequences shown in the present application or by the genes represented by these polynucleotide sequences. The term “fragment” en-closes therefore such fragments as primers for PCR, probes for hybridization, DNA fragments included in DNA vectors like plasmids, cosmids, BACs or viral constructs, as well as shortened splice variants of the genes identified herein. As a fragment of a protein (polypeptide) amino acid sequences are designated which have at least three amino acids, preferably at least 10 amino acids. Therefore fragments serving as antigens or epitopes are en-closed in this designation.

In the present application the term “sequence” is used when either a polynucleotide sequence (=nucleic acid sequence) or a polypeptide (=amino acid sequence) or a protein is meant. That means, when it is irrelevant which type of sequence is used the type is not designated particularly, but with the more common term “sequence”.

In the present application the term “stroke” means the development, occurrence, progression and consequences of the disease state. Several features of the development, occurrence and consequences of this disease are described herein above.

The basis of the methods and assays described in the present application is the examination of Cathepsin Y, which is differentially expressed under stroke. For the examination necessary for the development of any medicament each sequence can be used which allows the determination of the expression rate of the Cathepsin Y gene or the activity of the polypeptide/protein. The considered sequence can be the polynucleotide sequences SEQ ID NO: 1, SEQ ID NO: 2 or SEQ ID NO: 4, respectively or homologues or fragments thereof, as well as the polypeptides encoded thereby (SEQ ID NO: 3 or SEQ ID NO: 5, respectively) or homologues or fragments thereof.

According to the invention it has been found out, that the Cathepsin Y gene represented by the polynucleotide sequences SEQ ID No: 2 is differentially expressed in the cardiac arrest models for stroke, which is described above.

Therefore the present invention provides a target for the development of medicaments for the treatment of stroke, particularly for the treatment of ischemic stroke. Cathepsin Y has not yet been regarded in relation to stroke.

JP2000157263 describes a human Cathepsin Y useful in the elucidation and therapy for Alzheimer's disease, neurodegenerative diseases, cancers and the like. Comparing the nucleic acid sequence shown in this publication with the sequences of common databases, however, shows that the provided sequence corresponds to Cathepsin F, not to Cathepsin Y.

WO 96/39194 teaches that the Cathepsin Y protein is involved in the secretion of β-amyloid peptide (β-AP) from cells, resulting in the development of Alzheimer's disease. The nucleic acid sequence as well as the protein sequence of Cathepsin Y is provided. For the treatment of Alzheimer's disease the inhibition of this protein is proposed, therefore inhibitors of Cathepsin Y are examined.

According to the present invention the Cathepsin Y sequences (polynucleotide sequence or polypeptide) can be used in any test system which allows the determination of either the expression rate of the gene or the activity of the polynucleotide sequence or of the polypeptide/protein.

For determination and comparison of the expression levels of at least one of the sequences identified in the present invention any of the commonly known methods can be used, either on RNA/cDNA level or on protein level. For example PCR, hybridization, microarray based methods, Western blot or 2-D protein gel analysis are suitable methods. One preferred method is the digital expression pattern display method (DEPD method), explained in de-tail in WO99/42610. The method used for determination of expression levels is not restrictive, as long as expressed amounts can be quantified.

Activity of the Cathepsin Y protein can be determined by contacting the protein with polypeptides under acid pH conditions and determining the concentration of free amino acids after cleavage. Activity of the Cathepsin Y protein is a carboxypeptidase activity. Methods for determination of Cathepsin Y activity are explained in detail in international patent application WO 96/39194.

Expression and activity of Cathepsin Y can further be determined in assay systems or models. Such assay systems may be in vivo, ex vivo or in vitro assays, for example a cellular assay system comprising cells expressing Cathepsin Y, or an assay comprising isolated Cathepsin Y.

In any assay or model at least one of the sequences is contacted with the compound(s) to be tested and samples are obtained, wherein expression levels or activity of the sequences are determined and compared to non-treatment conditions.

For examination of the expression rate of the gene, animal models can be used. As such a model any animal can be used wherein the necessary preparations can be carried out, however, mammalian models are preferred. Even more preferred are rodents, particularly preferred are rats and mice. The most preferred animal model of the present invention is a rat model.

Dependent of the model used, the samples can be derived from whole blood, cerebrospinal fluid (CSF) or whole tissue, from cell populations isolated from tissue, cerebrospinal fluid (CSF) or blood or from single cell populations (i.e. cell lines).

In one embodiment of the invention cellular assays can be used. Preferred cells for cellular assays are eukaryotic cells, more preferably mammalian cells. Most preferred are neuronal-like cells, like SHSY5Y (neuroblastoma cell line), hippocampal murine HT-22 cells, primary cultures from astrocytes, cerebral cortical neuronal-astrocytic co-cultures, mixed neuronal/glial hippocampal cultures, cerebellar granular neuronal cell cultures, primary neuronal cultures derived from rat cortex (E15-17), or COS cells (African green monkey, kidney cells); CHO cells (Chinese hamster ovary), or HEK-293 cells (human embryonic kidney).

The Cathepsin Y sequences of the present invention can be used for diagnosing stroke, particularly ischemic stroke of a human outside of the living body, by determining the expression levels or activity of Cathepsin Y sequences in comparison to the non-disease status. During treatment period of a patient the expression or activity of the sequences can also be used for assessing the efficacy of stroke treatment outside of the body. In these cases blood, CSF or tissue is removed from the patient and expression or activity is determined in the samples.

The disease status, which is considered in the present invention, is stroke. The preferred types of stroke are ischemic and hemorrhagic stroke. Further diseases, which might be related to stroke, are severe headache, paralysis, cognitive deficits, speech problems, emotional difficulties, daily living problems, and central pain syndrome.

Independent whether the efficacy of stroke treatment or the efficiency of a compound for the treatment of stroke shall be examined or a stroke status is diagnosed or characterized, determination of the expression level or the activity of Cathepsin Y is carried out outside of a living body.

As mentioned above, detection of the expression of Cathepsin Y can be carried out by any method known in the art. The method of detection is not limiting the invention.

Expression levels can be detected either on basis of a polynucleotide sequence or by detecting the according polypeptide, encoded by said polynucleotide sequence.

Preferred methods for detection and determination of the gene expression levels are PCR of cDNA, generated by reverse transcription of expressed mRNA, hybridization of polynucleotides (Northern-, Southern Blot systems, In situ hybridization), DNA-microarray based technologies, detection of the according peptides or protein via, e.g., Western Blot systems, 2-dimensional gel analysis, protein microarray based technologies or quantitative assays like, e.g., ELISA tests.

The most preferred method for quantitative analysis of the expression levels is the differential expression pattern display method (DEPD), described in detail in WO99/42610.

By using any of the Cathepsin Y sequences (polynucleotide sequence or polypeptide/protein) the efficiency of compounds for reducing the expression of the sequence or the activity of the protein can be tested. Therefore the sequences of the present invention can be used for identifying therapeutic agents and their efficacy for the treatment of stroke.

For example, a method can be used comprising:

a) providing a test cell population comprising cells capable of expressing the Cathepsin Y gene(s) or homologues or fragments thereof

b) contacting said test cell population with the test therapeutic agent,

c) detecting the expression of the Cathepsin Y gene(s) in said test cell population,

d) comparing the expression of the gene(s) in the test cell population to the expression of the gene(s) in a reference cell population whose disease stage is known; and

e) identifying a difference in expression levels of the considered sequences, if present, in the test cell population and the reference cell population, thereby identifying a therapeutic agent for treating stroke.

For this method an animal model can be used or test cells can be obtained from a subject, an animal model or cell cultures of fresh cells or cell lines. Further in vitro assays may be used.

Agents or compounds, which have any influence on the expression rate of the Cathepsin Y gene(s) might be used as a medicament for the treatment of stroke. A method examining the expression rate of the Cathepsin Y gene can therefore be used for testing agents and compounds for their efficiency for treatment of stroke. Which model is used is not relevant, as long as the model allows determining differences in expression amounts.

In such a model cells are contacted with the interesting agent or compound, and expression of Cathepsin Y gene is determined in comparison to the expression in cells which have never been contacted by the according agent/compound. Contacting the cells with the agent/compound can either be achieved by administering the agent/compound to an animal, or to isolated cells of tissue or blood, or cell lines in culture.

By examination of the influence the considered agent/compound has to the expression of the Cathepsin Y gene the efficacy of the agent/compound for treating stroke can be estimated. This allows the decision whether it is worthwhile to develop a medicament containing such an agent or compound for the treatment of stroke.

Whether the expression is determined on the basis of mRNA generation (transcription level) or on basis of protein generation (translation level) is not relevant, as long as the difference of the expression rate can be determined. Therefore the polynucleotide sequence, as well as the polypeptide or protein shown in the present application can be used for the development of a medicament.

The development of a medicament can be desirable for example if the considered compound/agent has any influence on the regulation of the expression rate or on the activity of Cathepsin Y.

Said influence of a compound or agent can be examined by a method comprising contacting a sample comprising the nucleic acid sequence or homologues or fragment thereof or the according polypeptides of the present invention with a compound that binds to said sequence in an amount sufficient to determine whether said compound modulates the expression rate or the activity of the polynucleotide or polypeptide sequence.

By such a method a compound or agent can be determined modulating the expression rate or activity of the nucleic acid sequence or homologues or fragments thereof or the according polypeptides.

A method for determination of the efficiency of a compound/agent for the treatment of stroke by determination of the activity of the Cathepsin Y protein comprises for example contacting the Cathepsin Y protein with a compound/agent in presence of a known polypeptide which is a target of the Cathepsin Y, determining the amino-terminal amino acid of the peptide or the amount of free amino acids in the sample after incubation, comparing the amino-terminal amino acid of the peptide or the amount of free amino acids with the result in a sample which doesn't contain the compound/agent.

If a compound/agent is able to decrease or inhibit the activity of the Cathepsin Y protein it might be useful as a medicament for the treatment of stroke.

Examples for compounds, which can decrease Cathepsin Y activity, are compounds of formula I:

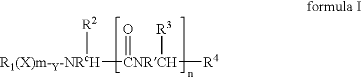

wherein:

R is selected from the group consisting of hydrogen, alkyl of from 1 to 6 carbon atoms, and where R and R² are joined to form a ring structure of from 4 to 10 carbon atoms,

R′ is selected from the group consisting hydrogen, alkyl of from 1 to 6 carbon atoms and where R″ and R3 are joined to form a ring structure of from 4 to 10 carbon atoms,

R₁ is selected from the group consisting of: alkyl of from 1 to 4 carbon atoms substituted with from 1 to 5 substituents selected from the group consisting of (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 car-bon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of: nitrogen, oxygen and sulfur, wherein said substituted alkyl group is optionally further substituted with from 1 to 2 hydroxyl groups, alkenyl of from 2 to 4 carbon atoms substituted with from 1 to 4 substituents selected from the group consisting of: (a) aryl of from 6 to 10 carbon atoms, (b) aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, (c) cycloalkyl of from 3 to 8 carbon atoms and (d) heterocycles of from 3 to 14 carbon atoms having from 1 to 3 heteroatoms selected from the group consisting of: nitrogen, oxygen and sulfur, aryl of from 6 to 10 carbon atoms, aryl of from 6 to 10 carbon atoms substituted with 1 to 3 substituents selected from the group consisting of alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, fluorenyl, heterocycles of from 3 to 14 carbon atoms having from 1 to 3 heteroatoms selected from the group consisting of nitrogen, oxygen and sulfur;

R² and R³ are independently D- or L-amino acid side chains of at least 2 carbon atoms with the proviso that said amino acid side chains do not include the proline side chain;

R⁴ is selected from the group consisting of —C(O)CH═N═N, —CH₂OH, —C═NOH, and —C(O)R⁵ where R⁵ is hydrogen, alkyl of from 1 to 6 carbon atoms, haloalkyl of from 1 to 6 carbon atoms and 1 to 2 halo groups, alkoxy of from 1 to 6 carbon atoms, —NR⁶R⁷ where R⁶ and R⁷ are independently selected from the group consisting of hydrogen and alkyl of from 1 to 6 carbon atoms, and aryl of from 6 to 10 carbon atoms, and —N(CH₃)OCH₃;

X is selected from the group consisting of —O—, —NR⁹—, and —S—, where R⁹ is selected from the group consisting of: hydrogen, alkyl of from 1 to 6 carbon atoms, and aryl of from 6 to 10 carbon atoms;

Y is selected from the group consisting of: —C(O)— and —C(S)—;

m is equal to zero or one; and

n is equal to zero, one or two,

or pharmaceutically acceptable salts thereof,

with the proviso that when R¹ is 1-naphthyl, R² is —CH(CH₃)₂ (L-isomer), R³ is —CH₂-Ø (L-isomer), Y is —C(O)—, m is zero and n is one, then R⁴ is not —N(CH₃)OCH₃,

with the further proviso that when R′ is diphenylmethyl, R² is p-(benzyloxy)benzyl (L-isomer), Y is —C(O)—, and m and n are zero, then R⁴ is not —N(CH₃)OCH₃, and

with still the further proviso that when R¹ is (1,2diphenyl)ethenyl, Y is —C(O)—, R² is —CH₂-Ø (L-isomer), and m and n are zero, then R⁴ is not —N(CH₃)OCH₃.

In a preferred embodiment

R′ includes benzyl, trityl, diphenylmethyl, 4phenylbutyl, 2-phenylethyl, naphthyl, pyridyl, fluorenyl, xanthanilyl, and the like.

R² and R³ are independent side chains of a D- or L- amino acid having at least 2 carbon atoms with the proviso that R² and R³ are not proline. Such side chains refer to the R⁸ substituent found on naturally occurring and synthetic amino acids of the formula H₂NCHR⁸COOH. Side chains of naturally occurring amino acids include, by way of example only, those where R⁸ is the L-isomer of (CH₃)₂CH— (valine), (CH₃)₂CHCH₂ (leucine), CH₃CH₂CH(CH₃)-(isoleucine), ØCH₂-(phenylalanine), (3-indolyl)-CH₂-(tryptophan), CH₃SCH₂CH₂-(methionine), CH₃CH(OH) (threonine), p-H-Ø-CH₂-(tyrosine), H₂NC(O)CH₂-(asparagine), H₂NC(O)CH₂CH₂-(glutamine), HOC(O)CH₂-(aspartic acid), HOC(O)CH₂CH₂-(glutamic acid), H₂NCH₂CH₂CH₂CH₂-(lysine), H₂NC(NH)NHCH₂CH₂CH₂-(arginine), 4-imidazolyl-CH₂-(histidine) and the like.

Side chains of synthetic amino acids include the D-isomer of the above noted naturally occurring amino acids as well as those where R⁸ is selected from the group consisting of alkyl of from 2 to 6 carbon atoms (where the alkyl group does not occur in naturally occurring amino acids), cycloalkyl of from 3 to 8 carbon atoms, and alkyl of from 1 to 6 carbon atoms substituted with from 1 to 2 substituents selected from the group consisting of:

aryl of from 6 to 10 carbon atoms,

aryl of from 6 to 10 carbon atoms substituted with from 1 to 3 substituents selected from the group consisting of: alkyl of from 1 to 6 carbon atoms, alkoxy of from 1 to 6 carbon atoms, aryl of from 6 to 10 carbon atoms, and aryloxy of from 6 to 10 carbon atoms, and heteroaryl of from 3 to 14 carbon atoms having from 1 to 3 heteroatoms selected from the group consisting of: nitrogen, oxygen and sulfur (where the substituted alkyl group does not occur in naturally occurring amino acids).

Particularly preferred amino acid side chains include the D- and L-isomers of valine, leucine, phenylalanine, tryptophan and isoleucine.

R⁴ preferably is —CH═N═N, or —C(O)H.

X preferably is —O—.

Y preferably is —C(O)—.

m and n preferably are 0 or 1.

In the present application -Ø means a phenyl residue.

Preferred compounds include, by way of example, the following compounds as defined by formula II below, including all isomers thereof, wherein the amino acid side chain for R² and R³ is indicated beneath the R² and R³ substituent:

formula II

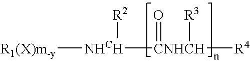

| R | ′X | m | Y | R² | n | R³ | R⁴ |
|---|---|---|---|---|---|---|---|
| Ø-CH₂— | O | 1 | —C(O)— | —CH(CH₃)₂ | 1 | —CH₂-Ø | —C(O)CH═N═N |
| | | | | (valine) | | (phenylalanine) | |
| Ø-CH₂— | 0 | 1 | —C(O)— | —CH₂-Ø | 1 | —CH₂-Ø | —C(O)CH═N═N |
| | | | | (phenylalanine) | | (phenylalanine) | |
| (Ø)₂-CH— | — | 0 | —C(O)— | —CH₂-Ø | 0 | — | —C(O)H |
| | | | | (phenylalanine) | | | |
| Ø-(CH₂)₄- | — | 0 | —C(O)— | —CH₂-Ø | 0 | — | —C(O)H |
| | | | | (phenylalanine) | | | |
| (Ø)₃-C— | — | 0 | —C(O)— | —CH₂-Ø | 0 | — | —C(O)H |
| | | | | (phenylalanine) | | | |
| (Ø)₂-CH— | — | 0 | —C(O)— | —CH₂—CH₂-Ø | 0 | — | —C(O)H |
| | | | | (homophenylalanine) | | | |
| ØCH═C( | — | 0 | —C(O)— | —CH₂-Ø | 0 | — | —C(O)H |
| Ø) | | | | (phenylalanine) | | | |
| (Ø)₂-CH— | — | 0 | —C(O)— | —CH₂-(3-indolyl) | 0 | — | —C(O)H |
| Ø-CH₂— | 0 | 1 | —C(O)— | —CH₂-Ø | 1 | —CH(CH₃)₂ | —C(O)H |
| | | | | (phenylalanine) | | (valine) | |
| Ø-CH₂— | 0 | 1 | —C(O)— | —CH(CH₃)₂ | 1 | —CH₂—CH(CH₃)₂ | —C(O)H |
| | | | | (valine) | | (leucine) | |

The term “heterocycles containing from 3 to 14 carbon atoms and 1 to 3 heteroatoms selected from the group consisting of nitrogen, oxygen and sulfur” refers to saturated and unsaturated heterocyclic groups having the requisite number of carbon atoms and heteroatoms. Suitable heterocyclic groups include, by way of example, furazanyl, furyl, imidazolidinyl, imidazolyl, imidazolinyl, indolyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, quinoxalinyl, thiadiazolyl, thiazolyl, thienyl, thiomorpholinyl (e.g., thiomorpholino), triazolyl, and xanthanilyl.

If in the present application a chemical group is described as containing for example “from 1 to 6 carbon atoms” or “from 6 to 10 carbon atoms” or “from 1 to 4 heteroatoms” it is meant that the group can contain either 1 or 2 or 3 or 4 or 5 or 6, or 6 or 7 or 8 or 9 or 10, or 1 or 2 or 3 or 4 of said atoms, respectively.

Heterocyclic groups can be substituted or unsubstituted. Where the heterocyclic group is substituted, the substituents are selected from alkyl of from 1 to 6 carbon atoms, alkoxy of from 1 to 6 carbon atoms, aryl of from 6 to 10 carbon atoms, aryloxy of from 6 to 10 carbon atoms, and halo.

Preferred heterocycles include well known cyclic aromatic groups containing heteroatoms within the cyclic structure. Such groups include, by way of example, furyl, imidazolyl, oxazolyl, pyrazolyl, pyridyl, pyrimidinyl, thiazolyl, and triazolyl.

The term “alkyl” refers to straight and branched chain alkyl groups such as methyl, ethyl, n-propyl, iso-propyl, n-butyl, tert-butyl, sec-butyl, iso-butyl, n-pentyl, n-hexyl, 2-methylpentyl, and the like; whereas the term “alkoxy” refers to -O-alkyl substituents.

The term “aryl” refers to aromatic substituents comprising carbon and hydrogen such as phenyl, naphthyl and the like whereas the term aryloxy refers to -O-aryl substituents where aryl is as defined above.

The term “halo” or “halogen” refers to fluorine, chlorine, bromine and iodine and preferably fluorine and chlorine.

The term “pharmaceutically acceptable salts” refers to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid.

Representative salts include the hydrochloride, hydrobromide, sulfate, bi-sulfate, acetate, oxalate, valerate, oleate, laurate, borate, benzoate, lactate, phosphate, tosylate, citrate, maleate, fumarate, succinate, tartrate, and napsylate salts, and the like. The particular salt employed is not critical.

Particularly preferred embodiments of the compounds usable as a medicament for the treatment of stroke by inhibiting protein activity of Cathepsin Y are the compounds 1 to 13 as shown in the following:

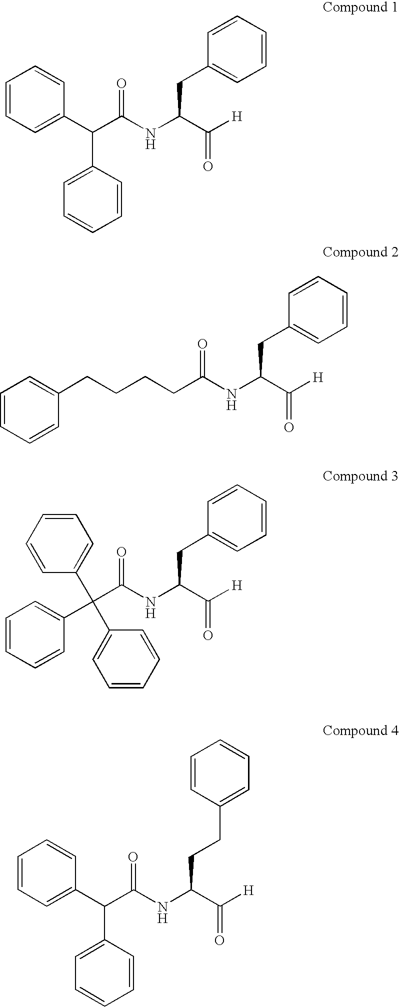

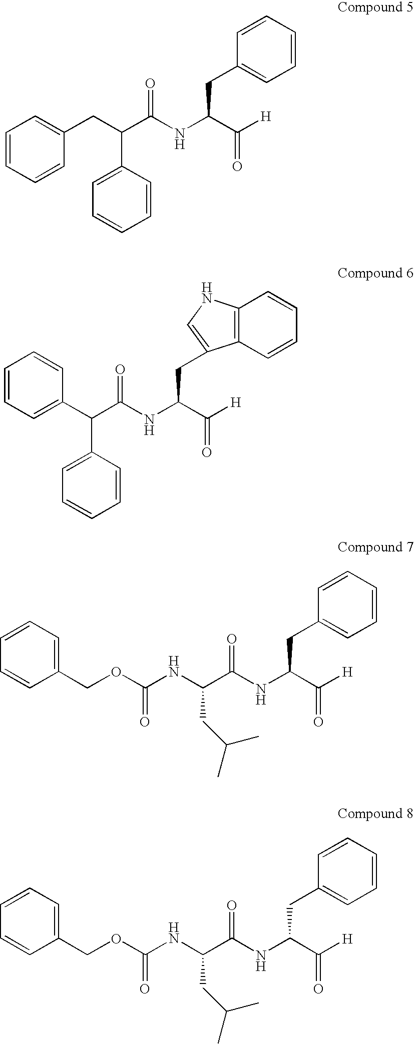

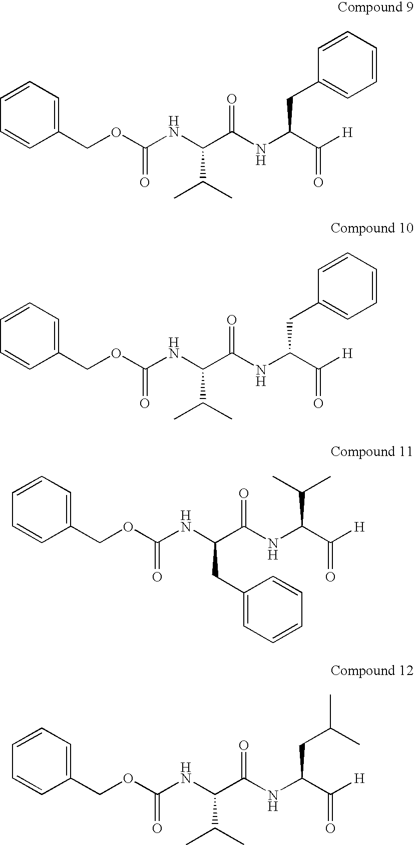

Preparation of the compounds 1 to 12 of the present application is explained in detail in WO96/39194.

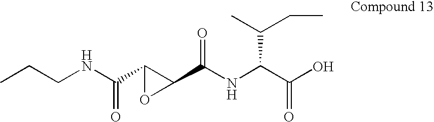

Preparation of compound 13 is explained in detail in the article of Therrien, C. et al. 2001 Biochemistry 40:2702-2711.

Furthermore the sequences of the present invention itself can be used as a medicament.

An example for such a use is the use of a polynucleotide sequence as an antisense agent. Antisense agents, including, but not limited to, ribonucleotide or desoxyribonucleotide oligomers, or base-modified oligomers like phosphothioates, methylated nucleotides, or PNAs (peptide nucleic acids), can hybridize to DNA or mRNA, inhibiting or decreasing transcription or translation, respectively. Thus, polynucleotide sequences of a gene, which is increased in expression rate under stroke, can be used as antisense agents to decrease the expression rates of said gene. Further such polynucleotide sequences can be used for gene therapy.

A pharmaceutical composition comprising a polynucleotide sequence according to the present invention can be any composition, which can serve as a pharmaceutical one. Salts or aids for stabilizing the sequence in the composition preferably are present.

For the determination of the expression of Cathepsin Y the generated sequence has to be detected. Therefore several reagents can be used, which are for example specific radioactive or non-radioactive (e.g. biotinylated or fluorescent) probes to detect nucleic acid sequences by hybridization, primer sets for the detection of one or several of the nucleic acid sequences by PCR, DNA microarrays, antibodies against one of the polypeptides, or epitopes, or antibody or protein microarrays. Such reagents can be combined in a kit, which can be sold for carrying out any of the de-scribed methods.

For further examinations or experiments it might be desirable to include the nucleic acid sequence of the present invention into a vector or a host cell. By including the sequence in a host cell, for example cellular assays can be developed, wherein the gene, polynucleotide sequences and the ac-cording protein/polypeptides can be used or examined.

Further the sequences defined in the present invention can be used to “design” new transgenic animals as models for stroke. Therefore the animals are “created” by manipulating the gene(s) encoding Cathepsin Y in a way that their expression in the transgenic animal differs from the expression of the same gene(s) in the wild type. “Manipulation” preferably results in a different expression level of the Cathepsin Y in the transgenic animal compared to the wild type, or in a protein with different enzymatic activity. Whether the expression level or the protein activity becomes higher or lower than the wild type under the same conditions depends on the desired model for stroke. Methods of gene manipulation and methods for the preparation of transgenic animals are commonly known to those skilled in the art.

The following examples are provided for illustration and are not intended to limit the invention to the specific example provided.

### EXAMPLE 1

### Preparation of Rat Cardiac Arrest Model

Cardiac arrest was performed in female Lewis-rats at 3 months of age (150-220 g), resulting in total cessation of blood flow leading to global cerebral ischemia. After 10 min of ischemia, the animals were resuscitated by external heart massage and ventilation. The group size was 2×3 animals.

For induction of cardiac arrest, a special blunt-end, hook-like probing device was inserted into the right parasternal line across the third intracostal spaces into the chest cavity. Next, the probe was gently pushed down the vertebral column until a slight resistance from the presence of the right pulmonary veins was detected. The probe then was tilted 10-20° caudally. Then, the probe was rotated in a counter-clockwise direction about 135-140° under the inferior vena cava. At this position, the occluding part of the device was positioned right under the heart vessel bundle (inferior vena cava, superior right and left vena cava, ascending aorta, and pulmonary trunk). The pulmonary veins (left and right) were closed by the rotation of the occluding part of the hook. In the last step, the probe was pulled up with concomitant compression of the heart vessel bundle against the sternum. The end of the occluding portion of the hook was then positioned in the left parasternal line in the second intercostal space. To prevent upward movement of the chest and to insure complete ligation of the vessels, simple finger pressure was applied downward the sternum, producing total hemostasis and subsequent ventricular arrest. The effect of the whole procedure is total cessation of both arterial and venous blood flow, it essentially represents the onset of clinical death. After 2.5-3.5 min, the probe was released and removed from the chest by reverse procedural succession, and the animals remained in this position until the beginning of resuscitation.

The resuscitation procedure consisted of external heart massage until spontaneous heart function was recovered and controlled respiration occurred. During this time, air was pumped through a polyethylene tube inserted intratracheally that was connected to a respirator. External heart massage was produced by the index and middle fingers rapidly striking against the chest (sternum) for 1-2 min at the level of the fourth intracostal area with a frequency of 150-240/min in continuous succession. The ratio of strikes to frequency of ventilation was 6:1 or 8:1.

An electrocardiogram (lead II-EEG) was recorded continuously during the course of the experiment. Moreover, heart activity was monitored using a loud speaker connected to the output lead of the electroencephalograph. Additionally, the cranial bones were exposed at the sagittal and coronal sutures, where silver-needle electrodes were attached for recording on an electrocorticogram (ECoG). All measurements were registered on a ten-channel electroencephalogram (Accutrace-100A, Beckmann).

2×3 sham-operated animals served as controls. These animals were treated similarly to the experimental group with one major exception. Under anesthesia, the probe was inserted through the chest wall into the plural cavity as has already been described above but without further manipulation and torsion of the probe. The probe remained in the chest for essentially the same time period (3.5 min) as in the experimental group. The control animals were then returned to their cages for recovery.

Tissue preparation occurred 0.5 h, 1 h, 3 hrs, 6 hrs, 3 days, 7 days and 2 years after surgery. Tissues were frozen on liquid nitrogen prior to RNA preparation.

### EXAMPLE 2

### Determination of Expression Levels

Gene expression profiling by DEPD-analysis starts with the isolation of 5-10 μg total RNA. In a second step, double-stranded cDNA is synthesized. Through an enzymatic digest of the cDNA with three different type IIS restriction enzymes, three pools with short DNA-fragments containing single-stranded overhangs are generated. Afterwards, specific DNA-adaptor-molecules are ligated and in two subsequent steps 3.072 PCR reactions are performed by using 1024 different unlabelled 5′ primer and a common FAM fluorescent labelled 3′-primer in the last PCR step. Subsequently, the 3072 PCR pools are analyzed on an automatic capillary electrophoresis sequencer.

Differential gene expression pattern of single fragments are determined by comparison of normalized chromatogram peaks from the control groups and corresponding operated animals.

### EXAMPLE 3

### Sequencing and Databank Analysis of the Obtained Sequences

Differentially expressed peaks are confirmed on polyacrylamide gels by using radioactive labelled 3′ primer instead of the FAM fluorescent primer. Differentially expressed bands are cut from the gel. After a short elution step in 60 μl 10 mM Tris pH 8, fragments are re-amplified by PCR using the same primer as used in the DEPD analysis. Resulting PCR products are treated with a mixture of Exonuclease I and shrimp alkaline phosphatase prior to direct sequencing. Sequencing reactions are performed by using a sequencing kit like DYE-namic ET dye terminator sequencing kit, and subsequently analyzed by capillary electrophoresis (Megabace 1000, Amersham).

Prior to a BLAST analysis (Altschul et al. 1997 Nucleic Acids Res 25:3389-3402) against Genbank (Release 126), sequence was quality verified and redundant sequences or repetitive motifs are masked.

Results are shown in Table 1 and are compared to human sequence.

| SEQ ID NO: | name | length |
|---|---|---|
| 1 | EST of Cathepsin Y (rat) | 255 bp |
| 2 | rattus norvegicus Cathepsin Y DNA | 1387 bp |
| 3 | rat Cathepsin Y protein | 306 aa |
| 4 | human Cathepsin Y DNA | 1500 bp |
| 5 | human Cathepsin Y protein | 303 aa |

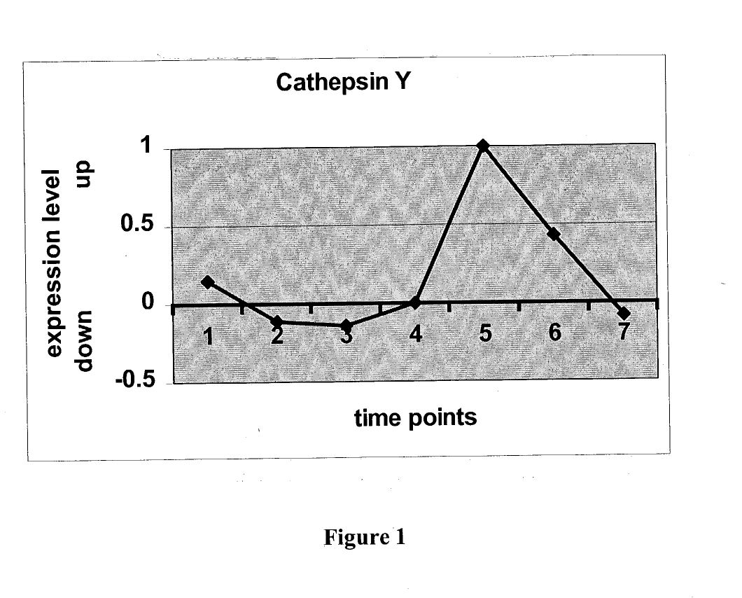

Expression of the Cathepsin Y gene in the stroke model over a time period of 0.5 h to 2 years is shown in FIG. 1.

### EXAMPLE 4

### Enzymatic Activity of Cathepsin Y Protein

Cathepsin Y does not have the standard endopeptidic activity associated with proteases. In an effort to identify whether the enzyme has other proteolytic activity, a number of randomly selected synthetic oligopeptides were incubated with purified Cathepsin Y at pH 5.5 or 4.5. Specifically, purified Cathepsin Y was incubated with the selected synthetic oligopeptides (50 μg/ml) at either pH 4.5 or pH 5.5, for 1 hour at 37° C. Samples were quenched by the addition of trifluoroacetic acid to 1% final concentration, then analyzed by reverse phase HPLC on a Vydac C18 column, using a gradient of increasing acetonitrile in 0.1% trifluoroacetic acid. Individual peaks (parent and new product(s)) were collected, then analyzed by acid hydrolysis followed by amino-acid analysis.

The results are summarized below:

| | |
|---|---|
| Parent Sequence | Product(s) |

| | | |
|---|---|---|
| LFYDQSPTATI | LFYDQSPTAT | (aa 1-10 of the parent sequence) |
| (SEQ ID NO:6) | LFYDQSPTA | (aa 1-9 of the parent sequence) |
| | LFYDQSPT | (aa 1-8 of the parent sequence) |
| YKRDMVGGVVIA | YKIRDMVGGVVI | (aa 1-11 of the parent sequence) |
| (SEQ ID NO:7) | YKRDMVGGVV | (aa 1-10 of the parent sequence) |
| EGYYGNYGVYA | EGYYGNYGVY | (aa 1-10 of the parent sequence) |
| (SEQ ID NO:8) | EGYYGNYGV | (aa 1-9 of the parent sequence) |
| FFDEPNPGVTIY | FFDEPNPGVT | (aa 1-10 of the parent sequence) |
| (SEQ ID NO: 9) | | |

The above peptides, as well as other peptides recited herein, are listed, per convention, from amino terminus to the carboxyl terminus.

The results from a number of such experiments (all not shown) suggest that the proteolytic activity of Cathepsin Y is a sequential removal of the car-boxy terminal amino acids, which is direct evidence for carboxypeptidase activity. No evidence of any endopeptidase or aminopeptidase activity was seen with any of these substrates. These data strongly suggest that the predominant proteolytic activity manifested by Cathepsin Y is carboxypeptidase activity.

The quantitative analysis of carboxypeptidase activity of Cathepsin Y is based on the detection of the new free amino-terminus generated on cleavage of a selected substrate. This is accomplished by reacting with the reagent ophthalaldehyde in an alkaline solution in the presence of 2-mercaptoethanol (Simons, et al. 1976 JACS 98:7098-7099). The peptide EGYYGNYGV was synthesized acetylated on its amino-terminus so that on cleavage by Cathepsin Y, the only free amino-terminus present in the reaction mixture will be that of the valine residue, cleaved off by the carboxypeptidase activity of the protease.

A standard curve was constructed by incubating varying concentrations of valine (0-20 μM) in 0.25 M sodium borate, pH 10, containing 0.05% 2-mercaptoethanol and 60 μg/ml o-phthalaldehyde, in individual wells of a 96 well microtiter plate. The resulting fluorescence is read in a plate reading Cytofluor (ex 340, em 460 nm). There is a linear increase in the signal proportional to the amount of free valine present.

In order to determine the pH optimum of the enzyme, reaction mixtures were set up with enzyme and substrate (0.2 mg/ml) in a total reaction volume of 0.1 ml) in individual wells of 96-well microtiter plates, in 20 mM sodium acetate buffers at different pHs, with 0.1% 2-mercaptoethanol present. Control wells were set identically except for the presence of enzyme. At various timepoints after incubation at room temperature, the reactions were quenched by the addition of an equal volume of 0.45 M sodium borate, pH 10, with 0.25 mg/ml o-phthalaldehyde, and the fluorescence measured. In the absence of added enzyme, no measurable fluorescence is generated above background, even with extended incubations. In the presence of enzyme, there is a time-dependent increase in fluorescence. Based on this analysis, the pH optimum for carboxypeptidase activity was determined to be about 4.5, with the activity dropping off at both lower and higher pHs.

Using this assay at pH 4.5, the ability of a number of compounds to inhibit the activity of Cathepsin Y was tested, by adding the desired concentration of the inhibitor in the incubation mixture along with enzyme and substrate, and measuring the decrease in fluorescence (if any) relative to enzyme control. Each of the compounds 1 to 13 tested in this analysis indicated inhibition of Cathepsin Y activity.

What is claimed is:

1. A method for identifying potential therapeutic agents for treating stroke, comprising:
a) providing a test cell capable of expressing a Cathepsin Y gene or homologues or fragments thereof;
b) contacting said test cell with the potential therapeutic agent;
c) detecting a level of expression of the Cathepsin Y gene in said test cell;
d) comparing the level of expression of the Cathepsin Y gene in the test cell to a level of expression of the Cathepsin Y gene in a reference cell whose disease stage is known; and
e) identifying a difference in the expression levels of the Cathepsin Y gene in the test cell and reference cell, thereby identifying the potential therapeutic agent for treating stroke.

2. The method of claim 1, wherein expression of the Cathepsin Y gene is determined by at least one method selected from the group consisting of PCR of a cDNA, hybridizing a sample DNA, and detecting a Cathepsin Y protein.

3. The method of claim 1, wherein said stroke is ischemic stroke.

4. A method for identifying a therapeutic agent for treating stroke, comprising:
a) incubating a sample comprising a Cathepsin Y protein, a test compound/agent, and a polypeptide which is a target of the Cathepsin Y protein for proteolysis;
b) determining an aminoterminal amino acid of a peptide resulting from the proteolysis of said target polypeptide or the amount of free amino acids in the sample after step (a);
c) comparing the aminoterminal amino acid of the peptide or the amount of free amino acids with a result obtained in a sample which does not contain the test compound/agent, thereby identifying the therapeutic agent for treating stroke.

5. The method of claim 4, wherein said stroke is ischemic stroke.

6. A pharmaceutical composition for the treatment of stroke, comprising a compound having a general formula: 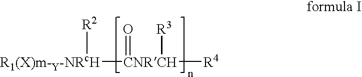
wherein:
R is selected from the group consisting of: hydrogen, and alkyl of from 1 to 6 carbon atoms, and where R and R² are joined to form a ring structure of from 4 to 10 carbon atoms,
R′ is selected from the group consisting of: hydrogen, and alkyl of from 1 to 6 carbon atoms, and where R′ and R³ are joined to form a ring structure of from 4 to 10 carbon atoms,
R₁ is selected from the group consisting of: alkyl of from 1 to 4 carbon atoms substituted with from 1 to 5 substituents selected from the group consisting of:
(a) aryl of from 6 to 10 carbon atoms,
(b) aryl of from 6 to 10 carbon atoms substituted with 1 to 3 substituents selected from the group consisting of: alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino,
(c) cycloalkyl of from 3 to 8 carbon atoms, and
(d) heterocycles of from 3 to 14 carbon atoms having from 1 to 3 heteroatoms selected from the group consisting of: nitrogen, oxygen and sulfur,
wherein said substituted alkyl group is optionally further substituted with a substituent selected from the group consisting of: 1 to 2 hydroxyl groups, alkenyl of from 2 to 4 carbon atoms substituted with a substituent selected from the group consisting of: 1 to 4 substituents selected from the group consisting of:
(a) aryl of from 6 to 10 carbon atoms,
(b) aryl of from 6 to 10 carbon atoms substituted with 1 to 3 substituents selected from the group consisting of: alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino,
(c) cycloalkyl of from 3 to 8 carbon atoms, and
(d) heterocycles of from 3 to 14 carbon atoms having from 1 to 3 heteroatoms selected from the group consisting of: nitrogen, oxygen and sulfur, aryl of from 6 to 10 carbon atoms, aryl of from 6 to 10 carbon atoms substituted with 1 to 3 substituents selected from the group consisting of: alkyl of from 1 to 6 carbon atoms, aryl of from 6 to 10 carbon atoms, alkoxy of from 1 to 6 carbon atoms, aryloxy of from 6 to 10 carbon atoms, hydroxy, cyano, halo and amino, fluorenyl, heterocycles of from 3 to 14 carbon atoms having from 1 to 3 heteroatoms selected from the group consisting of: nitrogen, oxygen and sulfur;
R² and R³ are independently D- or L-amino acid side chains of at least 2 carbon atoms, with the proviso that said amino acid side chains do not include the proline side chain;
R⁴ is selected from the group consisting of: —C(O)CH═N═N, —CH₂OH, —C═NOH, and —C(O)R⁵,
where R⁵ is hydrogen, alkyl of from 1 to 6 carbon atoms, haloalkyl of from 1 to 6 carbon atoms and 1 to 2 halo groups, alkoxy of from 1 to 6 carbon atoms, —NR⁶R⁷ where R⁶ and R⁷ are independently selected from the group consisting of: hydrogen and alkyl of from 1 to 6 carbon atoms, and aryl of from 6 to 10 carbon atoms, and —N(CH₃)OCH₃;
X is selected from the group consisting of: —O—, —NR⁹—, and —S—, where R⁹ is selected from the group consisting of: hydrogen, alkyl of from 1 to 6 carbon atoms and aryl of from 6 to 10 carbon atoms;
Y is selected from the group consisting of —C(O)— and —C(S)—;
m is equal to zero or one; and
n is equal to zero, one or two,
or pharmaceutically acceptable salts thereof,
with the proviso that when R¹ is 1-naphthyl, R² is —CH(CH₃)₂ (L-isomer), R³ is —CH2-Ø (L-isomer), Y is —C(O)—, m is zero and n is one, then R⁴ is not —N(CH₃)OCH₃,
with the further proviso that when R′ is diphenylmethyl, R² is p-(benzyloxy)benzyl (L-isomer), Y is —C(O)—, and m and n are zero, then R⁴ is not —N(CH₃)OCH₃, and
with still the further proviso that when R¹ is (1,2diphenyl)ethenyl, Y is —C(O)—, R² is —CH2-Ø (L-isomer), and m and n are zero, then R⁴ is not —N(CH₃)OCH₃.

7. The pharmaceutical composition of claim 6, wherein the compound is an inhibitor of Cathepsin Y protein activity.

8. The composition of claim 6, wherein the compound is selected from the group consisting of:

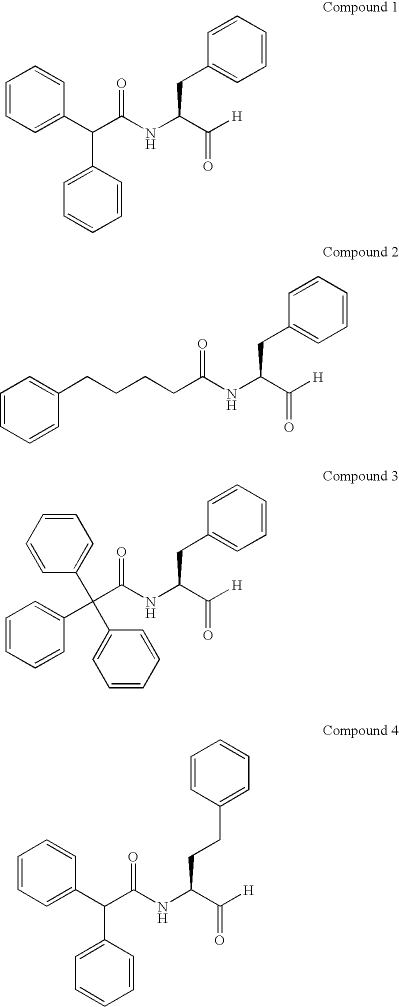

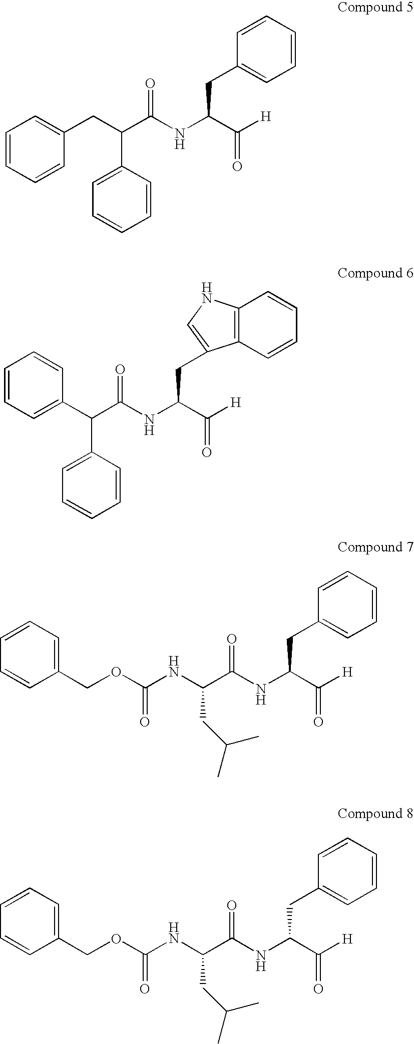

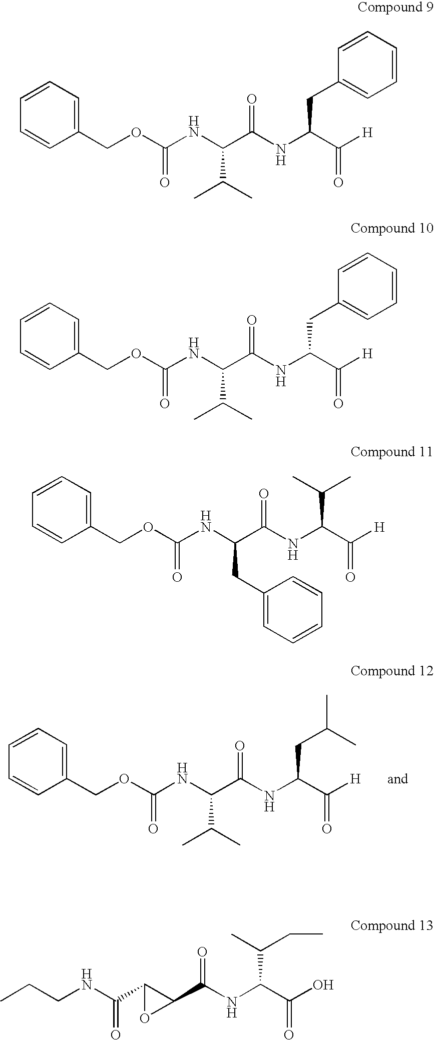

9. The composition of claim 6, wherein the stroke is ischemic stroke.

10. The composition of claim 6, wherein the stroke is hemorrhagic stroke.

11. The composition of claim 7, wherein the stroke is ischemic stroke.

12. The composition of claim 7, wherein the stroke is hemorrhagic stroke.

13. The composition of claim 8, wherein the stroke is ischemic stroke.

14. The composition of claim 8, wherein the stroke is hemorrhagic stroke.

15. A pharmaceutical composition for the treatment of stroke, comprising a nucleic acid sequence which is an “antisense” sequence compared to a nucleic acid sequence encoding Cathepsin Y of SEQ ID NO: 2, or SEQ ID NO 4, or homologues or fragments thereof.

16. The composition of claim 15, wherein said stroke is ischemic stroke.

17. The composition of claim 15, wherein said stroke is hemorrhagic stroke.
